(19) United States
(12) Patent Application Publication
(10) Pub. No.: US 2011/0054441 A1
(43) Pub. Date: Mar. 3, 2011

(54) METHODS, DEVICES, AND COMPOSITIONS FOR INTRAVITREAL INJECTION

(76) Inventors: SIGNE R. ERICKSON, Menlo Park, CA (US); Charles J. Hagemeier, Laguna Beach, CA (US)

(21) Appl. No.: 12/853,781

(22) Filed: Aug. 10, 2010

Related U.S. Application Data
(60) Provisional application No. 61/232,711, filed on Aug. 10, 2009.

(51) Int. Cl. A61F 9/007

(52) U.S. Cl. 604/506

(57) ABSTRACT

Methods of treating disorders of the eye are disclosed. One or more substances are injected into the vitreous humor of the eye using a syringe. A needle of the syringe is inserted into the eye such that the tip of the needle is positioned inferior to the visual axis. The needle of the syringe is inserted into the eye at an injection point that is located from 3 mm to 5 mm posterior to the limbus of the eye. The tip of the needle is positioned at a depth from 1 mm to 10 mm from the retina of the eye at the injection point.

### CROSS-REFERENCE TO RELATED PATENT APPLICATION

This application claims the benefit of U.S. Provisional Application No. 61/232,711, filed on Aug. 10, 2009, which is hereby incorporated by reference in its entirety.

### FIELD

This invention relates to methods for treating disorders of the eye and, more particularly, to methods for treating disorders of the eye by injecting substances into the eye.

### BACKGROUND

Most drugs in development and approved for treating “back of the eye” diseases are injected directly into the vitreous humor, a thick clear gel that fills the space between the lens and retina. To date, the focus of the injection technique has centered around prevention of infection, and little work has been done regarding the location and formulation of the injected material. The importance in controlling the distribution of injected materials in the eye has become particularly apparent when delivering microparticle formulations. Without controlling the injection procedure and other formulation variables, these particles can float into the visual field over time, or adhere to other ocular tissues. To address the safety and efficacy of these systems, more control over distribution is needed.

Injection techniques, surgical instrumentation, and formulation variables all play roles in controlling the initial location of injected material in the eye. These factors have been refined herein to limit the migration and distribution of injected material over time. Key advantages of the disclosed methods, devices, and compositions include maintaining therapeutic material proximal to the disease site and preventing adverse effects, such as obstruction of the visual field and interaction with and damage to the retina and lens.

### SUMMARY

The invention relates to methods of treating disorders of the eye by injecting a substance into the vitreous humor of the eye using a syringe. The syringe has a barrel containing the substance, a needle having a tip and a lumen in fluid communication with the barrel, and a plunger that is movable toward and away from the needle within the barrel. In one embodiment, the method comprises inserting the needle into the eye at an injection point positioned along an arc centered on the visual axis of the eye. The arc extends from a first point on the temporal side of the eye about 30° (degrees) above an imaginary horizontal plane containing the visual axis to a second point on the nasal side of the eye about 30° (degrees) above the imaginary horizontal plane. The needle is injected to a depth within the eye such that the tip of the needle is positioned below the imaginary horizontal plane. The method further comprises moving the plunger toward the needle to thereby force the substance from the barrel through the lumen and into the vitreous humor of the eye.

In another embodiment, the method comprises inserting the needle into the eye through the pars plana at an injection point positioned inferior to the visual axis of the eye. The needle is inserted to a depth such that the tip of the needle is positioned inferior to the visual axis. The method further comprises moving the plunger toward the needle to thereby force the substance from the barrel through the lumen and into the vitreous humor of the eye.

In an additional embodiment, the method comprises identifying an injection point on the surface of the pars plana of the eye. The injection point is positioned along an arc centered on the visual axis of the eye. The arc extends from a first point on the temporal side of the eye about 30° (degrees) above an imaginary horizontal plane containing the visual axis to a second point on the nasal side of the eye about 30° (degrees) above the imaginary horizontal plane. The injection point is located 3 to 5 mm posterior to the limbus of the eye. The method further comprises orienting the needle at an orientation angle 90° (degrees) to 45° (degrees) relative to an imaginary line tangent to the injection point. The imaginary line tangent to the injection point intersects the visual axis. The method further comprises inserting the needle into the eye at the orientation angle through the injection point. The needle is injected into the eye to a depth within the eye such that the tip of the needle is positioned below the imaginary horizontal plane. The depth of the tip of the needle within the eye is from 1 mm to 10 mm from the retina at the injection point. The method still further comprises moving the plunger toward the needle to thereby force the substance from the barrel through the lumen and into the vitreous humor of the eye.

### DETAILED DESCRIPTION OF THE FIGURES

These and other features of the preferred embodiments of the invention will become more apparent in the detailed description in which reference is made to the appended drawings wherein:

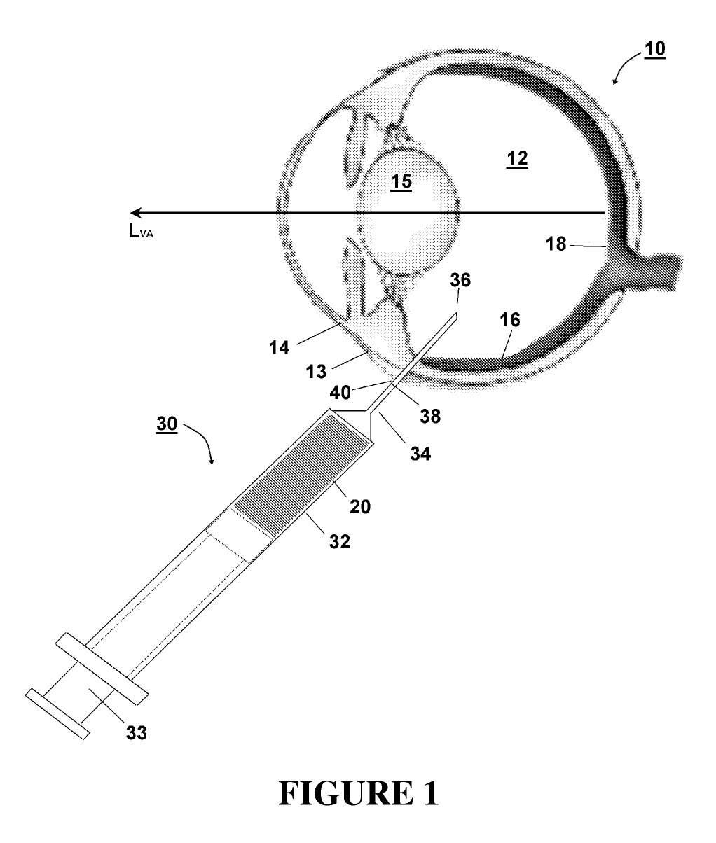

FIG. 1 depicts the injection of a substance into the eye according to the methods described herein.

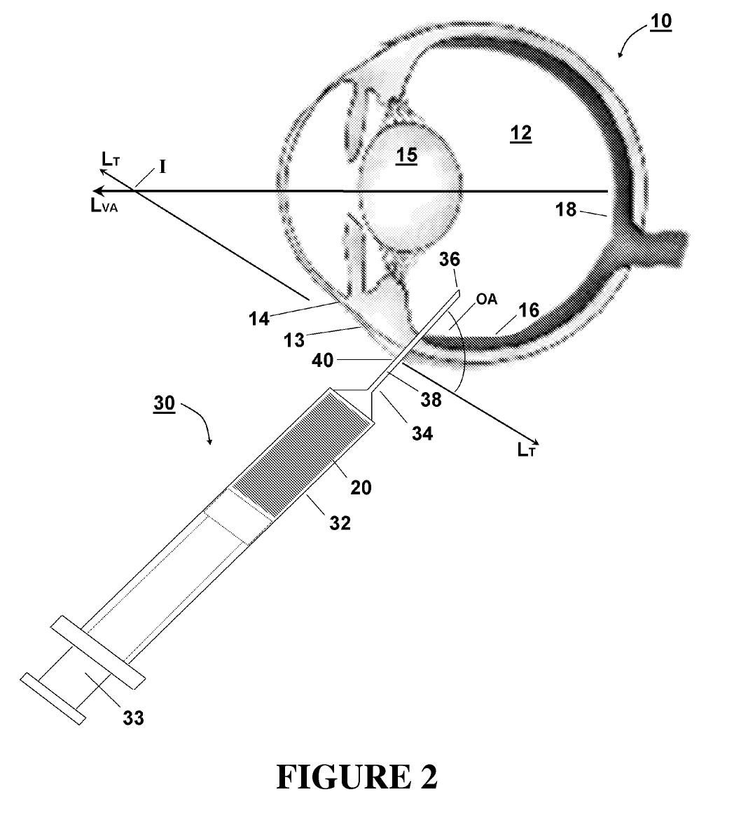

FIG. 2 depicts the orientation of a needle at an orientation angle according to the methods described herein.

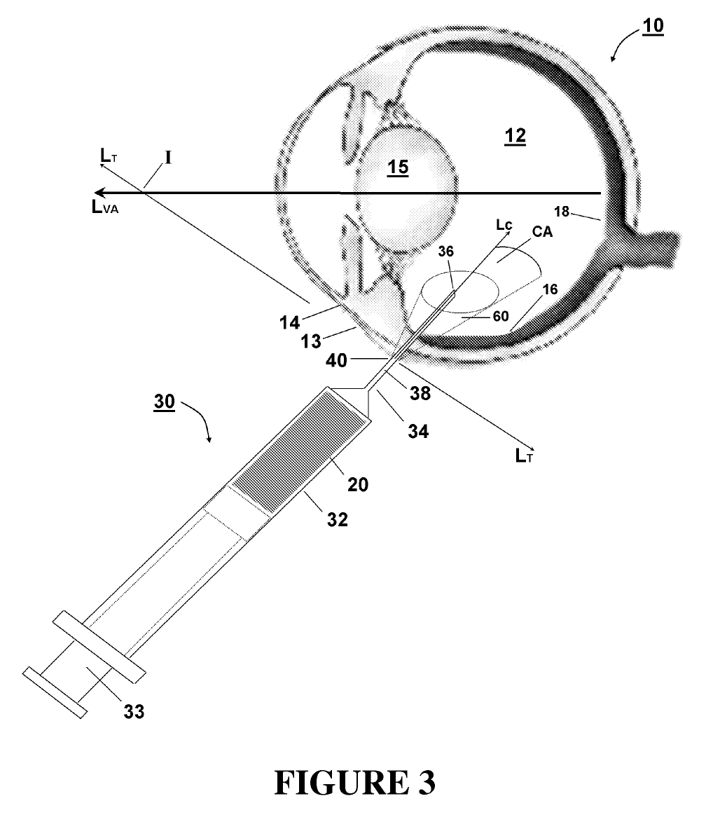

FIG. 3 depicts the orientation of a needle within a cone within the eye according to the methods described herein.

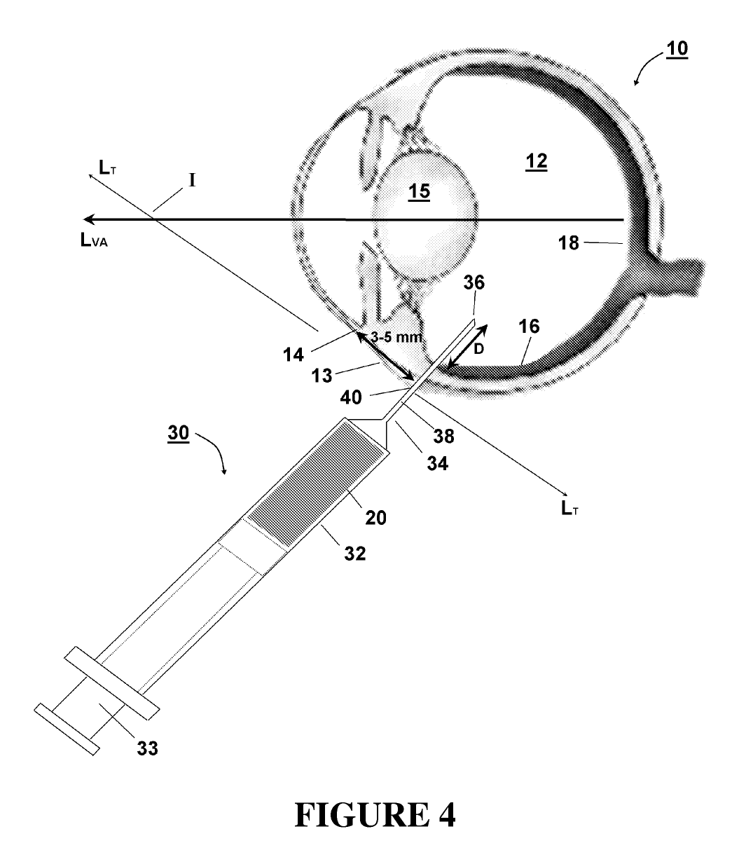

FIG. 4 depicts the positioning of a needle and an insertion point for insertion of the needle according to the methods described herein.

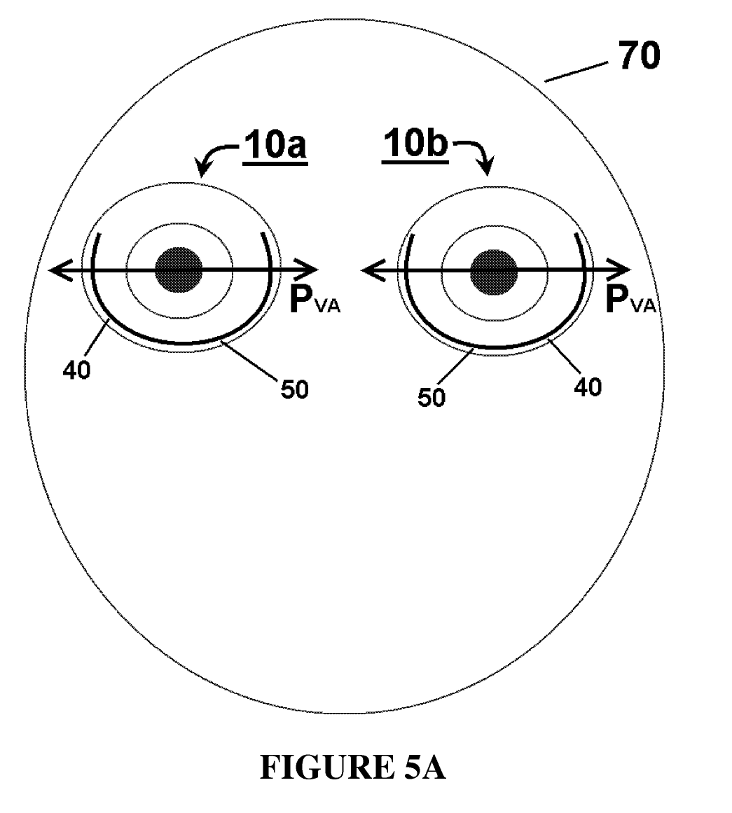

FIG. 5A depicts an arc on which an injection point is located according to the methods described herein. FIG. 5B depicts an arc on which the injection point is more preferably located according to the methods described herein. FIGS. 5A and 5B are not to scale.

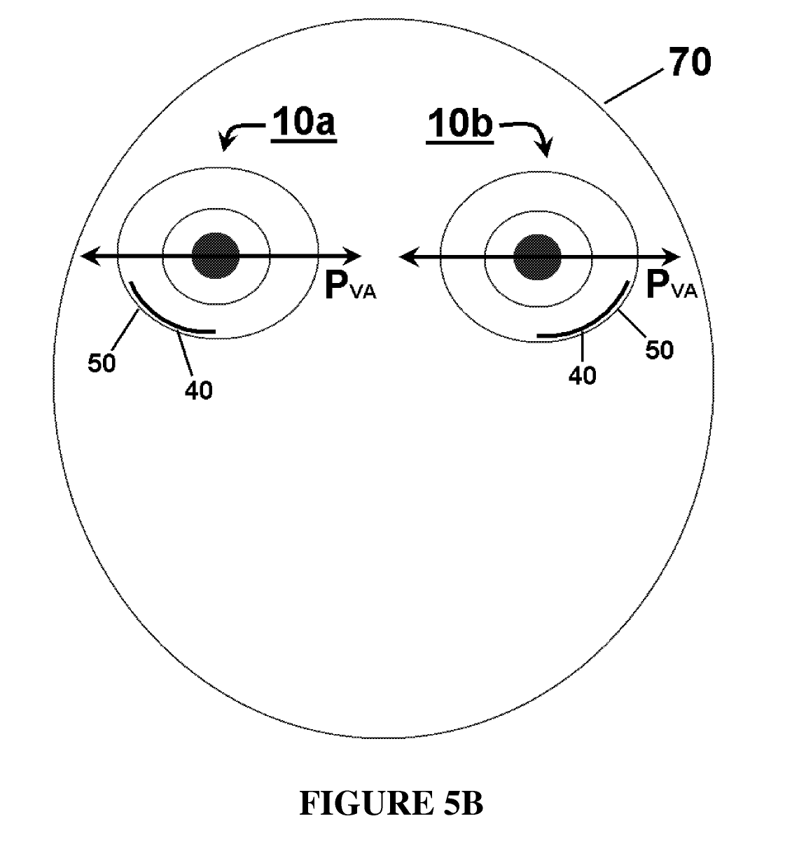

FIG. 6 depicts a side view of an eye that has received an injection of a substance according to the methods described herein.

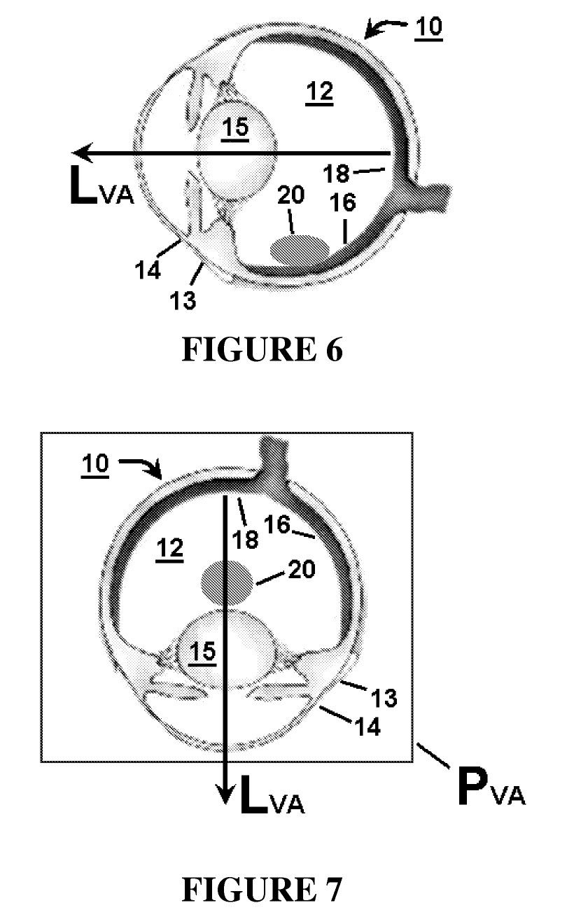

FIG. 7 depicts a top view of the eye depicted in FIG. 6.

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

Before the present methods, microparticles, compounds, compositions, and/or devices are disclosed and described, it is to be understood that the aspects described herein are not limited to specific compounds, synthetic methods, or uses as such can, of course, vary. It is also to be understood that the terminology used herein is for the purpose of describing particular aspects only and, unless specifically defined herein, is not intended to be limiting.

In this specification and in the claims that follow, reference will be made to a number of terms that shall be defined to have the following meanings:

As used throughout, the singular forms “a,” “an” and “the” include plural referents unless the context clearly dictates otherwise. Thus, for example, reference to “a needle” can include two or more such needles unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein, a “wt. %” or “weight percent” or “percent by weight” of a component, unless specifically stated to the contrary, refers to the ratio of the weight of the component to the total weight of the composition in which the component is included, expressed as a percentage.

“Excipient” is used herein to include any compound or additive that is not a therapeutically or biologically active compound. As such, an excipient should be pharmaceutically or biologically acceptable or relevant (for example, an excipient should generally be non-toxic to the subject). “Excipient” includes a single such compound and is also intended to include a plurality of excipients.

The term “microparticle” is used herein to include nanoparticles, microspheres, nanospheres, microcapsules, nanocapsules, and particles, in general. As such, the term microparticle refers to particles having a variety of internal structure and organizations including homogeneous matrices such as microspheres (and nanospheres) or heterogeneous core-shell matrices (such as microcapsules and nanocapsules), porous particles, multi-layer particles, among others. The term “microparticle” refers generally to particles that have sizes in the range of about 10 nm (nanometers) to about 2 mm (millimeters).

“Subject” is used herein to refer to any target of administration. The subject can be a vertebrate, for example, a mammal. Thus, the subject can be a human. The term 10 does not denote a particular age or sex. Thus, adult and newborn subjects, as well as fetuses, whether male or female, are intended to be covered. A “patient” refers to a subject afflicted with a disease or disorder and includes human and veterinary subjects.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination of molecules, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be 30 considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Disclosed herein, and as shown in FIGS. 1-4, are methods for treating a disorder of an eye 10 of a subject by injecting a substance 20 into the vitreous humor 12 of the eye. In one aspect, the substance 20 can be injected into the vitreous humor 12 of the eye 10 using a syringe 30. In this aspect, the syringe 30 can have a barrel 32 configured to contain the substance 20 prior to injection. In another aspect, the syringe 30 can have a needle 34. In this aspect, the needle 34 can have a tip 36 and a lumen 38 in fluid communication with the barrel 32 of the syringe. It is contemplated that the needle 34 can be metallic. It is further contemplated that the tip 36 of the needle 34 can be sharpened or otherwise configured for introduction into the eye 10. The needle 34 can have any diameter that is suitable for introduction into the eye 10, and thus, can be any gauge that is suitable for introduction into the eye, including, for example and without limitation, 20, 21, 22, 23, 24, 25, 26, 27, 28, 29, 30, 31, 32, 33, and 34 gauge. In an additional aspect, the syringe 30 can have a plunger 33. In this aspect, the plunger 33 can be movable toward and away from the needle 34 within the barrel 32. It is contemplated that, after the needle 34 is placed in fluid communication with the substance 20, the plunger 33 can be moved away from the needle to draw a desired amount of the substance into the barrel 32 of the syringe 30. After the substance 20 is contained within the barrel 32 of the syringe 30, any air trapped in the barrel 32 between the plunger 33 and the needle 34 can be purged or otherwise removed using conventional methods. Although the injection steps of the methods disclosed herein are generally accomplished with the use of a syringe, it is contemplated that the disclosed methods can also be accomplished using any other conventional injection mechanism, including, for example and without limitation, a pump injection mechanism, positive displacement piston rods, hydraulic injection mechanisms, and the like.

In one aspect, and as shown in FIGS. 5A and 5B, the methods of treating a disorder of the eye can comprise inserting the needle 34 into the eye 10 at an injection point 40 positioned along an arc 50 centered on the visual axis L_{VA} of the eye. As shown on the face 70 depicted in FIGS. 5A and 5B, the arc 50 can be positioned on either a right eye 10a or a left eye 10b. In this aspect, and as shown in FIG. 5A, it is contemplated that the arc 50 can extend inferiorly from a first point 52 on the temporal side of the eye 10a, 10b about 30° (degrees) above an imaginary horizontal plane P_{VA} containing the visual axis L_{VA} of the eye, to a second point 54 on the nasal side of the eye about 30° (degrees) above the imaginary horizontal plane. As used herein, the term “nasal side” refers to the side of the eye that is most proximate the subject's nose, while the term “temporal side” refers to the side of the eye that is most proximate the temple and, therefore, is opposed from the nasal side of the eye. Thus, the arc 50 can begin at a point 30° (degrees) above the imaginary horizontal plane P_{VA}, continue through the portion of the eye 10a, 10b below the imaginary horizontal plane, and terminate at a point 30° (degrees) above the imaginary horizontal plane. In illustrating the location of the arc 50 on the eye 10a, 10b, it is helpful to visualize a clock face that is superimposed on a front view of the eye. In this illustration, the arc 50 as described herein can extend from a point corresponding to the 2 o'clock position of the clock to a point corresponding to the 10 o'clock position of the clock.

In an additional aspect, the injection point 40 can be positioned on the arc 50 between a point located on the temporal side of the eye 10a, 10b substantially within the imaginary horizontal plane P_{VA} and a point located on the nasal side of the eye substantially within the imaginary horizontal plane. In this aspect, and in continuing the previous illustration, the injection point 40 can be positioned on the arc 50 between points corresponding to the 3 o'clock and 9 o'clock positions of the clock. In another aspect, the injection point 40 can be positioned on the arc 50 between a point located about 30° (degrees) below the imaginary horizontal plane P_{VA} on the temporal side of the eye 10a, 10b and a point located about 30° (degrees) below the imaginary horizontal plane on the nasal side of the eye. In this aspect, the injection point 40 can be positioned on the arc 50 between points corresponding to the 4 o'clock and 8 o'clock positions of the clock. In still another aspect, the injection point 40 can be positioned on the arc 50 between a point located about 90° (degrees) below the imaginary horizontal plane P_{VA} on the temporal side of the eye (the 6 o'clock position of the clock) and a point about 30° (degrees) below the imaginary horizontal plane P_{VA} on the nasal side of the eye (the 8 o'clock position of the clock for the left eye and the 4 o'clock position of the clock for the right eye). More preferably, and as shown on the face 70 depicted in FIG. 5B, the injection point 40 can be positioned on the arc 50 between a point located about 30° (degrees) below the imaginary horizontal plane P_{VA} on the temporal side of the eye (the 4 o'clock position of the clock for the left eye and the 8 o'clock position of the clock for the right eye) and a point located about 90° (degrees) below the imaginary horizontal plane on the temporal side of the eye (the 6 o'clock position of the clock).

In another aspect, and with reference to FIGS. 1-4, the arc 50 can overlie at least a portion of the pars plana 13 of the eye 10. In this aspect, it is contemplated that the arc 50 can overlie the entire pars plana 13 of the eye 10. In a further aspect, and with reference to FIG. 4, the arc 50 can be located from about 3 mm to about 5 mm posterior to the limbus 14 of the eye 10. More preferably, the arc 50 can be located from about 3 mm to about 4 mm posterior to the limbus 14 of the eye 10. In this aspect, it is contemplated that the arc 50 can be concentric with the limbus 14 of the eye 10. Thus, it is contemplated that the arc 50 and the limbus 14 can both be centered on the visual axis L_{VA} of the eye 10.

In a further aspect, and with reference to FIG. 2, the methods can comprise orienting the needle 34 at an orientation angle OA from about 90° (degrees) to about 45° (degrees) relative to an imaginary line L_{T} tangent to the surface of the eye 10 at the injection point 40. More preferably, the orientation angle OA can be from about 90° (degrees) to about 85° (degrees) relative to the imaginary line L_{T} tangent to the surface of the eye 10 at the injection point 40. Most preferably, the orientation angle OA can be from about 87° (degrees) to about 85° (degrees) relative to the imaginary line L_{T} tangent to the surface of the eye 10 at the injection point 40. It is contemplated that the imaginary line L_{T} tangent to the surface of the eye 10 can extend in any direction. Thus, the needle 34 can be oriented in any direction relative to the injection point 40. Optionally, in one aspect, the imaginary line L_{T} can intersect the visual axis L_{VA} of the eye at an intersection point I. In an additional aspect, it is contemplated that the needle 34 can be oriented at the orientation angle OA before the step of inserting the needle into the eye 10. Alternatively, the needle 34 can be oriented at the orientation angle OA after the step of inserting the needle into the eye 10.

In one aspect, and with reference to FIG. 3, it is contemplated that the methods can comprise orienting the needle 34 within an imaginary cone 60 positioned within the eye 10. In this aspect, the cone 60 can have a vertex coincident with the injection point 40. In an additional aspect, the cone can have a cone angle CA of about 45 degrees measured from a line L_{c} oriented perpendicular to the surface of the eye 10 at the injection point 40.

In another aspect, and with reference to FIG. 4, it is contemplated that the needle 34 can be inserted into the eye 10 at the injection point 40 to a depth D within the eye such that the tip 36 of the needle is positioned below the imaginary horizontal plane P_{VA}. In this aspect, the depth D of the tip 36 of the needle 34 within the eye 10 can be from about 1 mm to about 10 mm from the retina 16 at the injection point 40. More preferably, the depth D of the tip 36 of the needle 34 within the eye 10 can be from about 1 mm to about 4 mm from the retina 16 at the injection point 40.

In an additional aspect, and as shown in FIGS. 1-4, the methods can comprise moving the plunger 33 toward the needle 34, thereby forcing the substance 20 from the barrel 32 through the lumen 38 and into the vitreous humor 12. In one aspect, it is contemplated that the needle 34 can be selectively moved to create a pocket within the vitreous humor 12 for receipt of the substance 12 from the barrel 32 of the syringe 30. Thus, after the substance 20 exits the barrel 32 of the syringe 30 and enters into the vitreous humor 12, it is contemplated that the needle 34 can be removed from the vitreous humor while concurrently allowing the substance to remain within the vitreous humor. As depicted in FIGS. 1, 6 and 7, it is further contemplated that the substance 20 can settle downward within the vitreous humor 12 such that the substance avoids contacting the macula 18 and the lens 15 within the eye 10, thereby avoiding interference with the visual field of the subject.

In some aspects, it is contemplated that injection guides and injection assistance devices can be coupled with the syringes and other conventional injection mechanisms to perform the steps of the methods disclosed herein. It is further contemplated that the injection guides and injection assistance devices can be used to ensure that the substance is injected at a desired depth, angle, and position. Accordingly, it is contemplated that the syringes and other injection mechanisms disclosed herein can be coupled to, for example, and without limitation, gauges for measuring depth of injection, gauges for measuring angle of injection, guides for stabilizing injection, guides for controlling positioning of an injection, and the like. In one aspect, it is contemplated that the syringe can be coupled to an InVitria® Intravitreal Injection Assistant manufactured by FCI Ophthalmics (Pembroke, Mass.).

The disclosed methods can be used to treat or prevent a variety of disorders of the eye, including both anterior and posterior ocular conditions. In one aspect, the methods can be used to treat macular degeneration and abnormal macular angiogenesis, which can be associated with retinal edema and retinal neovascularization.

In other aspects, the methods can be practiced or provided to treat one or more disorders of the posterior segment of a mammalian eye, including, for example and without limitation, macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome.

In additional aspects, the methods can be used to treat one or more vascular conditions and disorders of the eye, including, for example and without limitation, retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease.

In further aspects, the methods can be used to treat traumatic/surgical conditions and disorders, including, for example and without limitation, sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis, and myiasis.

In other aspects, the methods can be used to treat genetic conditions and disorders, including, for example and without limitation, retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum;

In additional aspects, the disclosed methods can also be used to treat retinal diseases associated with cancer and tumors, including, for example and without limitation, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors.

In still further aspects, the methods can be used to treat or repair a wide range of ocular conditions, including, for example and without limitation, punctuate inner choroidopathy, acute posterior multifocal placoid pigment epitheliopathy, myopic retinal degeneration, acute retinal pigment epithelitis, retinitis pigmentosa, proliferative vitreal retinopathy (PVR), age-related macular degeneration (ARMD), diabetic retinopathy, diabetic macular edema, retinal detachment, retinal tears, uveitus, macular tears, cytomegalovirus retinitis, glaucoma, and conditions involving ocular degeneration, such as neurodegeneration of retinal ganglion cells.

In one aspect, the substance that is injected into the eye can comprise microparticles. In this aspect, it is contemplated that the substance that is injected into the eye can comprise from about 1 to about 500 mg of microparticles suspended in an injection vehicle. More preferably, the substance can comprise from about 2 to about 300 mg of microparticles suspended in an injection vehicle. Most preferably, the substance can comprise from about 3 to about 150 mg of microparticles suspended in an injection vehicle. The injection vehicle, in one aspect, can comprise from about 1% to about 50% solids. More preferably, the injection vehicle can comprise from about 10% to about 40% solids. Most preferably, the injection vehicle can comprise from about 20% to about 30% solids. In one exemplary aspect, the substance that is injected into the eye can comprise from about 10 mg to about 50 mg of microparticles suspended in an injection vehicle comprising from about 20% to about 30% solids. In use, the substances disclosed herein are typically injected directly into the vitreous humor in volumes from about 10 to about 150 μL per injection.

In another aspect, the microparticles that can be used in the disclosed methods can have an average or mean particle size from about 10 μm to about 125 μm. More preferably, the microparticles can have a mean particle size from about 20 μm to about 90 μm. Most preferably, the microparticles can have a mean particle size from about 30 μm to about 80 μm. It is contemplated that the particle size distributions disclosed above can be measured by laser diffraction techniques known to those of skill in the art.

In a further aspect, the microparticles can be prepared using one or more drug compositions. In this aspect, the drug compositions can comprise one or more water soluble carriers or excipients. It is contemplated that such carriers or excipients can generally include sugars, saccharides, polysaccharides, surfactants, buffer salts, bulking agents, viscosity agents, and the like. A non-limiting example of an excipient is 2-(hydroxymethyl)-6-[3,4,5-trihydroxy-6-(hydroxymethyl)tetrahydropyran-2-yl]oxy-tetrahydropyran-3,4,5-triol, “trehalose.” In one aspect, the drug composition can comprise from about 1 wt % to about 200 wt % trehalose based on the weight of trehalose in the starting drug composition. More preferably, the drug composition can comprise from about 10 wt. % to about 50 wt. % trehalose based on the weight of trehalose in the starting drug composition. Most preferably, the drug composition can comprise from about 25 wt % to about 35 wt % trehalose based on the weight of trehalose in the starting drug composition.

In another aspect, the excipient can comprise one or more surfactants, including, for example and without limitation, polysorbate 20, polysorbate 80, and the like. In one exemplary aspect, the excipient can comprise polysorbate 20 (or Tween 20). In this aspect, the drug composition can comprise from about 0.01 wt % to about 5 wt % polysorbate 20 based on the weight of polysorbate 20 in the starting drug composition. More preferably, the drug composition can comprise from about 0.05 wt % to about 0.25 wt % polysorbate 20 based on the weight of polysorbate 20 in the starting drug composition. Most preferably, the drug composition can comprise about 0.1 wt % polysorbate 20 based on the weight of polysorbate 20 in the starting drug composition. It is contemplated that the drug composition can comprise two or more carriers and/or excipients as described herein. For example, and without limitation, the drug composition can comprise from about 25 wt % to about 35 wt % trehalose and about 0.1 wt % polysorbate 20 based on the weights of the individual drugs in the starting drug composition.

In an additional aspect, the excipient can comprise one or more viscosity agents, including, for example and without limitation, hydroxypropyl methylcellulose (HPMC), hyaluronic acid, and the like.

Optionally, a conventional wetting or friction-reducing additive can be added to the substance to increase the wettability or lubricity of the substance. It is contemplated that these additives can be configured to promote the downward movement of the substance following injection of the substance into the eye.

In one aspect, the disclosed substances can be injected as described herein pursuant to a desired dosage schedule. For example, and without limitation, the desired dosage schedule can comprise a dose about every month, about every two months, about every three months, every four months, about every six months, about every eight months, about every nine months, and about every twelve months.

### EXPERIMENTAL EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric.

### Example 1

A range of injection techniques were investigated to control microparticle distribution. Specifically, coumarin-loaded microspheres with HPMC and Healon injection vehicles (50 μL) were injected into intact cadaveric porcine eyes (Sierra Medical) through a 25 gauge UTW needle. For optimal initial placement, the speed of injection was not critical. A shallow needle injection appeared to be ideal. During injection, needle movement was avoided to minimize the tendency of injected particles to follow channels and planes created by the needle. Air bubbles within the composition were minimized to prevent particles from being carried upwardly by the air bubbles within vitreous humor. Injections were located inferior to the visual axis to promote early settling of the injected particles in an inferior location.

### Example 2

The polymer system tolerability in the eye following intravitreal injection was evaluated. Additionally, the injection technique and impact of the system variables (particle size, dose mass, injection vehicle, and injection location) on microparticle distribution over time were evaluated. Microparticle sizes of <10, 10-32, 32-63 and >63 μm were tested. Dose mass was varied among 3, 10, and 20 mg. Diluted Healon (2000 kD, rooster comb) and HA Genzyme (500 kD, fermented) were tested as injection vehicles. Poly(lactide-co-glycolide) placebo microspheres were evaluated as microparticles within the injection vehicle. A single 50 μL injection was made into the eye for the 3 and 10 mg doses, while two 50 μL injections were made into the eye for the 20 mg dose.

Five groups of non-pigmented New England White rabbits were used in a bilateral dosing study. Ophthalmic examinations (including fundus exams, photography, and intraocular pressure measurements) were performed pre-operation, and at days 1, 8, 15, 31, 61, 91, and 180 (for Groups D-E) post-operation. Electroretinography (ERG) and Optical Coherence Tomography (OCT) analyses were performed pre-operation, and at day 180 for Groups D-E. At the end of the study (90 days for Groups A-C, 180 days for Groups D-E), histopathology samples were collected and analyzed.

Superior placement of injections resulted in significant presence of the injected particles in the visual field. In contrast, inferior placement of injections resulted in minimal presence of the injected particles in the visual field, and the number of inferiorly injected particles that were present within the visual field decreased significantly faster than the superiorly injected particles that were present within the visual field. Additionally, deep, inferior placement of injections led to settling of particles out of the visual field within three days. After settling, the particles dispersed at the base of the eye. In contrast, superior placement of injections generally led to slower settling of particles out of the visual field (within 90 days). Overall, for inferiorly placed injections, there was generally little change in location of particles up to 60 days post-operation, with particles remaining stable outside of the visual field. Degradation of the inferiorly injected particles was evident between 60 and 180 days post-operation.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A method of treating a disorder of an eye by injecting a substance into the vitreous humor of said eye using a syringe, said syringe having a barrel containing said substance, a needle having a tip and a lumen in fluid communication with said barrel, and a plunger movable toward and away from said needle within said barrel, said method comprising:
inserting said needle into said eye at an injection point positioned along an arc centered on the visual axis of said eye and extending inferiorly from a first point on the temporal side of said eye about 30° above an imaginary horizontal plane containing said visual axis, to a second point on the nasal side of said eye about 30° above said imaginary horizontal plane, to a depth within said eye such that said tip of said needle is positioned below said imaginary horizontal plane; and
moving said plunger toward said needle thereby forcing said substance from said barrel through said lumen and into said vitreous humor.

2. The method according to claim 1, wherein said arc overlies the pars plana of said eye.

3. The method according to claim 1, wherein said arc is located from about 3 mm to about 5 mm posterior to the limbus of said eye, said arc being concentric with said limbus.

4. The method according to claim 1, wherein said injection point is positioned on said arc between a third point located on the temporal side of said eye substantially within said imaginary plane and a fourth point located on the nasal side of said eye substantially within said imaginary plane.

5. The method according to claim 1, wherein said injection point is positioned on said arc between a third point located on the temporal side of said eye about 30° below said imaginary plane and a fourth point located on the temporal side of said eye about 90° below said imaginary plane.

6. The method according to claim 1, further comprising orienting said needle at an orientation angle from about 90° to about 45° relative to an imaginary line tangent to the surface of said eye at said injection point.

7. The method according to claim 6, wherein said imaginary line intersects said visual axis.

8. The method according to claim 6, wherein said needle is oriented at said orientation angle before inserting said needle.

9. The method according to claim 6, wherein said orientation angle is from about 90° to about 85° relative to said imaginary tangent line.

10. The method according to claim 6, wherein said orientation angle is from about 87° to about 85° relative to said imaginary tangent line.

11. The method according to claim 1, wherein said depth of said tip within said eye is from about 1 mm to about 10 mm from the retina at said injection point.

12. The method according to claim 1, further comprising orienting said needle within an imaginary cone positioned within said eye, said cone having a vertex coincident with said injection point.

13. The method according to claim 12, wherein said cone has a cone angle of about 45 degrees measured from a line oriented perpendicular to the surface of said eye at said injection point.

14. The method according to claim 1, wherein said substance comprises microparticles.

15. A method of treating a disorder of an eye by injecting a substance into the vitreous humor of said eye using a syringe, said syringe having a barrel containing said substance, a needle having a tip and a lumen in fluid communication with said barrel, and a plunger movable toward and away from said needle within said barrel, said method comprising:
inserting said needle into said eye through the pars plana at an injection point positioned inferior to the visual axis of said eye to a depth such that said tip of said needle is positioned inferior to the visual axis;
moving said plunger toward said needle thereby forcing said substance from said barrel through said lumen and into said vitreous humor.

16. The method according to claim 15, wherein said injection point is located from about 3 mm to about 4 mm posterior to the limbus of said eye.

17. The method according to claim 15, wherein said injection point is located on an arc centered on the visual axis of said eye, said arc extending inferiorly from a first point located on the temporal side of said eye about 30° below an imaginary horizontal plane containing the visual axis, to a second point located on the nasal side of said eye about 30° below said imaginary horizontal plane.

18. The method according to claim 15, wherein said injection point is located on an arc centered on the visual axis of said eye, said arc extending inferiorly from a first point located on the temporal side of said eye about 30° below an imaginary horizontal plane containing the visual axis, to a second point located on the temporal side of said eye about 90° below said imaginary horizontal plane.

19. The method according to claim 15, wherein said injection point is located on an arc centered on the visual axis of said eye, said arc extending superiorly from a first point located on the temporal side of said eye about 90° below an imaginary horizontal plane containing the visual axis, to a second point located on the nasal side of said eye about 30° below said imaginary horizontal plane.

20. The method according to claim 15, further comprising orienting said needle at an orientation angle from about 90° to about 45° relative to an imaginary line tangent to the surface of said eye at said injection point.

21. The method according to claim 20, wherein said imaginary line intersects said visual axis.

22. The method according to claim 20, wherein said needle is oriented at said orientation angle before inserting said needle.

23. The method according to claim 20, wherein said orientation angle is from about 90° to about 85° relative to said imaginary tangent line.

24. The method according to claim 20, wherein said orientation angle is from about 87° to about 85° relative to said imaginary tangent line.

25. The method according to claim 15, wherein said depth of said tip within said eye is from about 1 mm to about 10 mm from the retina at said injection point.

26. The method according to claim 15, further comprising orienting said needle within an imaginary cone positioned within said eye, said cone having a vertex coincident with said injection point.

27. The method according to claim 26, wherein said cone has a cone angle of about 45 degrees measured from a line oriented perpendicular to the surface of said eye at said injection point.

28. The method according to claim 15, wherein said substance comprises microparticles.

29. A method of treating a disorder of an eye by injecting a substance into the vitreous humor of said eye using a syringe, said syringe having a barrel containing said substance, a needle having a tip and a lumen in fluid communication with said barrel, and a plunger movable toward and away from said needle within said barrel, said method comprising:
identifying an injection point on the surface of the pars plana of said eye, wherein said injection point is positioned along an arc centered on the visual axis of said eye and extending inferiorly from a first point on the temporal side of said eye about 30° above an imaginary horizontal plane containing said visual axis, to a second point on the nasal side of said eye about 30° above said imaginary horizontal plane, and wherein said injection point is located from about 3 mm to about 5 mm posterior to the limbus of said eye;
orienting said needle at an orientation angle from about 90° to about 45° relative to an imaginary line tangent to said injection point, wherein said imaginary line intersects said visual axis;
inserting said needle into said eye at said orientation angle through said injection point to a depth within said eye such that said tip of said needle is positioned below said imaginary horizontal plane, wherein said depth of said tip within said eye is from about 1 mm to about 10 mm from the retina at said injection point; and
moving said plunger toward said needle thereby forcing said substance from said barrel through said lumen and into said vitreous humor.

30. The method according to claim 29, wherein said substance comprises microparticles.
